(19) United States
(12) Patent Application Publication
(10) Pub. No.: US 2025/0152093 A1
(43) Pub. Date: May 15, 2025

(54) MOBILE OR PORTABLE GAS MEASURING DEVICE AND PROCESS FOR DETERMINING THE CONCENTRATION OF NITROUS OXIDE AND ETHANOL IN A BREATH SAMPLE FROM A TEST SUBJECT

(71) Applicant: Dräger Safety AG & Co. KGaA, Lübeck (DE)

(72) Inventors: Stefan MORLEY, Lübeck (DE); Christine WESTPHAL, Lübeck (DE)

(21) Appl. No.: 18/939,804

(22) Filed: Nov. 7, 2024

(30) Foreign Application Priority Data
Nov. 9, 2023 (DE) 10 2023 131 130.1

(51) Int. Cl. A61B 5/00; A61B 5/08; A61B 10/00; G01N 33/497

(52) U.S. Cl. CPC A61B 5/4845; A61B 5/082; A61B 10/00; G01N 33/4972; G01N 33/4975; A61B 2010/0009; A61B 2010/0087

(57) ABSTRACT

A mobile or portable gas measuring device for determining the concentration of nitrous oxide and ethanol in a breath sample of a test person is provided. The mobile gas measuring device has an optical gas sensor for determining a nitrous oxide concentration in the breath sample and an electrochemical gas sensor for determining an ethanol concentration in the breath sample.

### CROSS REFERENCE TO RELATED APPLICATIONS

This application claims the benefit of priority under 35 U.S.C. § 119 of German Application 10 2023 131 130.1, filed Nov. 9, 2023, the entire contents of which are incorporated herein by reference.

### TECHNICAL FIELD

The present invention relates to a mobile or portable gas measuring device (breathalyzer) for determining a concentration of nitrous oxide and ethanol in a breath sample (breath gas sample/respiratory gas sample) of a test subject (test person), and to a process for determining the concentration of nitrous oxide and ethanol in a breath sample of a test person.

### BACKGROUND

Driving under the influence of drugs is one of the greatest risks for serious accidents, sometimes resulting in death. A major challenge for the police is detecting drug use in road traffic during traffic checks and after accidents. In addition to established and sufficiently detectable illegal substances, more and more substances are emerging that have shown a strong increase in consumption and present the police with major challenges in detecting consumption and influence.

One of these substances is nitrous oxide, also known as laughing gas. Today, the police cannot detect, determine or prove the consumption of nitrous oxide. This applies in particular to mobile on-site detection. A device for determining the concentration of nitrous oxide in the breath of test subjects using a hand-held gas measuring device is not available.

Devices for the expiratory measurement of anesthetic gases are generally known in the field of anesthesia. In particular, infrared optical measurement processes are used for this. In this respect, a corresponding gas analyzer is known from DE 196 28 310 C2. This consists of a radiation source, a measuring section that receives the gas sample and a pyroelectric detector, which is connected to an evaluation circuit. The concentrations of the respective components can be determined by filtering using interference filters in the beam path, the transmission wavelengths of which are matched to the absorption wavelengths of the gas components to be detected. However, such devices are bulky and have a high energy consumption, which makes them unsuitable for mobile use.

There is currently no known mobile or portable device, no breathalyzer, for the simultaneous measurement of nitrous oxide and ethanol in the exhaled air of a test person, so that emergency services have no means of dealing with people who are obviously under the influence of nitrous oxide.

### SUMMARY

It is therefore an object of the present invention to provide a mobile or portable gas measuring device and a corresponding process which meet this need.

This problem is solved by mobile or portable gas measuring device/breathalyzer with features according to the invention and by a process for determining a concentration of nitrous oxide and ethanol in a breath gas sample of a subject with features according to the invention. The description, the figures and the claims disclose advantageous embodiments of the invention.

According to the invention, a mobile or portable gas measuring device for determining the concentration of nitrous oxide and ethanol in a breath sample of a test person is provided. The mobile gas measuring device has an optical gas sensor for determining a nitrous oxide concentration in the breath sample and an electrochemical gas sensor for determining an ethanol concentration in the breath sample.

In this way, a gas measuring device is provided that enables the measurement, i.e. determination of the concentration, of both nitrous oxide and ethanol in a breath sample from a test person. As the gas measuring device is mobile or portable, it is possible for emergency services such as the police to take and analyze samples directly on site without having to transport the breath sample to a laboratory.

A mobile or portable gas measuring device (breathalyzer) is understood to be a gas measuring device for single measurements or continuous measurements which can be carried from place to place by human power and used while being carried, preferably while being held. A gas measuring device is understood to be a device which is configured for the measurement of at least two gas concentrations, namely for the measurement of a concentration of nitrous oxide and a concentration of ethanol in the test subject's breath sample.

A breath sample is a quantity of gas which a person, namely the test person, exhales and which is thus made available for measurement by means of the gas measuring device.

An optical gas sensor is understood to be a sensor which, by means of an optical measuring process, is able to provide a number of signals which indicate a concentration of a target gas, in particular nitrous oxide, in the gas or gas mixture to be analyzed, in particular in the breath sample. The optical measuring process is preferably configured as a spectroscopic measuring process, in particular as an infrared spectroscopic measuring process.

An electrochemical gas sensor is understood to be an electrochemical cell which is configured to detect at least one gaseous substance, in particular ethanol, in a gas or gas mixture, in particular in the breath sample, and to provide a number of measurement signals corresponding to the concentration of the substance.

Preferably, the electrochemical sensor comprises a measuring electrode and a counter electrode, preferably also a reference electrode. The presence of further and/or other electrodes is possible. Preferably, the electrochemical sensor comprises an acidic, liquid electrolyte which is in contact with at least some of the electrodes. The electrochemical gas sensor can be configured as a fuel cell.

Preferably, the gas measuring device also has a sample guide element (sample feed element) which can be connected to the optical gas sensor and the electrochemical gas sensor in terms of flow in order to guide the breath sample to the optical gas sensor and the electrochemical gas sensor.

The sample guide element can be used to direct the breath sample to the gas sensors.

A sample guide element is understood to be a component or an assembly, i.e. a plurality of components, which is or are suitable for receiving the breath sample from the test person and directing it in the direction of the optical gas sensor and the electrochemical gas sensor.

The sample guide element can be configured to intake (receive) the breath sample directly or indirectly.

Direct intake refers to the collection of the subject's breath sample without any further aids between the subject and the sample guide element, such as the subject blowing into the sample guide element.

Indirect intake refers to the intake of the subject's exhaled air using other means between the subject and the sample guide element, e.g. by collecting the breath sample in a sample container such as a bag and feeding this breath sample from the sample container into the sample guide element.

Preferably, the optical gas sensor has an optical cuvette, whereby the optical cuvette can optionally be heated.

The cuvette can be used such that the absorption path of the optical gas sensor can be specifically predetermined and separated from the rest of the gas measuring device.

In the optional case that the optical cuvette can be heated (with an integrated heater), it is also possible to thermally condition the breath sample in order to improve the measuring behavior of the gas measuring device. This can also prevent the formation of condensation in the optical cuvette.

Preferably, the optical gas sensor also has: a radiation source and a radiation detector. Optionally, the optical gas sensor can also have one or more further radiation detectors.

According to the invention, a radiation source refers to a component which is configured to emit radiation comprising ultraviolet radiation and/or infrared radiation.

According to the invention, ultraviolet radiation is understood to mean electromagnetic radiation with a wavelength in a range from 10 nm to 400 nm, preferably in a range from 100 nm to 380 nm.

According to the invention, infrared radiation (from the radiation source) is understood to mean electromagnetic radiation with a wavelength in a range from 0.750 μm to 1000 μm.

The wavelength used in the invention or a wavelength range used in the invention may be predetermined depending on the target gas or gases to be detected.

The radiation source according to the invention can emit the radiation in a broadband manner—for example, in that the radiation source is configured as an incandescent lamp—or in a narrowband manner, for example, in that the radiation source is configured as a light-emitting diode or as a laser diode.

According to the invention, a radiation detector is understood to be a component which is configured to detect radiation in the ultraviolet and/or infrared wavelength range, in particular in the range of the measurement wavelength. For this purpose, the radiation detector has at least one detector element.

The detector element of the radiation detector can, for example, be configured as a semiconductor detector, a pyroelectric detector, a thermoelectric detector or a thermal detector. A design as a CCD or CMOS sensor is also possible. The detector element is configured to provide a corresponding detector signal.

If the optical gas sensor has a radiation detector and a further radiation detector, it is possible to measure a further gas component of the breath sample in addition to the nitrous oxide concentration in the breath sample, in particular carbon dioxide.

Preferably, the sample guide element is configured as a mouthpiece.

In this way, compatibility can be achieved with mouthpieces already available for existing hand-held alcohol measuring devices, which can improve the handling of the gas measuring device according to the invention. The provision of a mouthpiece also facilitates sampling.

Preferably, the gas measuring device also has a pump unit which is configured to convey at least part of the breath sample to the optical gas sensor and/or to the electrochemical gas sensor.

In this way, the response time of the gas measuring device can be improved, as the breath sample to be analyzed—compared to a gas measuring device without a pump unit—is available for measurement more quickly at the respective gas sensor. Furthermore, the pump unit makes it possible to control a volume flow through the respective gas sensor so that the measurement behavior of the respective gas sensor can be specifically influenced.

In this respect, a pump unit is understood to be a component of the gas measuring device which is configured to convey at least part of the breath sample.

Preferably, the gas measuring device also has a further pump unit, which is configured to convey at least a further part of the breath sample to the electrochemical gas sensor and/or to the optical gas sensor. The pump units or pump unit may be provided as a pump arrangement. The pump arrangement comprises the pump unit configured and arranged to convey at least a portion of the breath sample to the electrochemical gas sensor and/or to the optical gas sensor or comprises a pump unit configured and arranged to convey at least a portion of the breath sample to the electrochemical gas sensor and a further pump unit configured convey at least a portion of the breath sample to the optical gas sensor.

The provision of a further pump unit has comparable advantages to the provision of a pump unit. If both a pump unit and the further pump unit are provided, it is also possible to influence the temporal course of the breath sample delivery in the direction of the respective gas sensor even more specifically, for example by assigning the pump unit to the optical gas sensor in terms of flow and by assigning the further pump unit to the electrochemical gas sensor in terms of flow.

Preferably, the pump unit and/or the further pump unit is configured as a bellows pump. The advantage of providing a bellows pump is that it can be integrated into a mobile or portable gas measuring device in a compact and cost-effective manner. A pump unit comprises at least one bellows. Several bellows can be driven by a common drive unit.

The pump unit is particularly preferably configured for continuous delivery of at least part of the breath sample to the optical gas sensor, preferably through its optical cuvette. Such a pump unit can, for example, be configured as a vane pump, a diaphragm pump or a piezo pump.

If the further pump unit is configured as a bellows pump, this offers the advantage that a predetermined volume of the further part of the breath sample is delivered to the electrochemical gas sensor, so that calibration of the electrochemical gas sensor is simplified.

Preferably, the optical gas sensor has a measurement wavelength in the range from 4.44 μm to 4.65 μm, particularly preferably in the range from 4.5 μm to 4.6 μm.

In the course of the invention, it was recognized that such a measurement wavelength is particularly suitable for measuring nitrous oxide.

Preferably, the optical gas sensor has an optical filter for selecting the measurement wavelength, whereby the optical filter can be replaceable (interchangeable) in order to adapt the optical gas sensor to different target gases.

In this way, the gas measuring device can measure further and/or other target gases in addition to or instead of nitrous oxide. For example, ethanol can be detected instead of nitrous oxide by changing the optical filter.

An optical filter is a component of the gas measuring device that selects the incident radiation according to predetermined criteria. Such a filter can, for example, be configured as a bandpass filter or as a double bandpass filter and select the radiation according to a wavelength or according to one or more wavelength ranges. The optical filter can, for example, be configured as an absorption filter or as a dielectric filter. The filter can be configured as a constant or tunable filter. A tunable filter offers the advantage that when only one radiation detector is provided, it is possible to switch between two wavelengths to be measured or between two wavelength ranges to be measured, for example in order to alternately measure a concentration of nitrous oxide and a concentration of carbon dioxide in the breath sample or in the part of the breath sample of the test person.

According to the invention, a process for determining the concentration of nitrous oxide and ethanol in a breath sample of a test person is also provided.

The process comprises the steps of: Providing a gas measuring device (breathalyzer) as described above, supplying at least a portion of the breath sample to the optical gas sensor, optionally obtaining a reference value which indicates a carbon dioxide concentration in the breath sample, determining a nitrous oxide concentration based on measurement signals from the optical gas sensor and optionally taking the reference value into account, providing the determined nitrous oxide concentration, supplying at least a further portion of the breath sample to the electrochemical gas sensor, determining an ethanol concentration based on measurement signals from the electrochemical gas sensor and providing the determined ethanol concentration.

The nitrous oxide concentration is the concentration of nitrous oxide in the breath sample.

The ethanol concentration refers to the concentration of ethanol in the breath sample.

Preferably, the reference value is a predetermined reference value. Alternatively, it is preferred that the reference value is obtained by measuring the carbon dioxide concentration in the breath sample using the gas measuring device.

The predetermined reference value can, for example, correspond to a usual carbon dioxide concentration of human subjects, e.g. a concentration of 200 ppm. Since nitrous oxide and carbon dioxide can exhibit cross-interference, for example if the measurement is made in a wavelength range in which both nitrous oxide and carbon dioxide absorb radiation, it is possible to compensate for the influence of carbon dioxide on the determined nitrous oxide concentration by taking the reference value into account. In a simple example, this can be done by reducing a provisionally determined nitrous oxide concentration by the carbon dioxide concentration indicated by the reference value. For example, if a preliminary nitrous oxide concentration of 300 ppm is calculated, this can be reduced by the carbon dioxide concentration indicated by the reference value, for example by 200 ppm, in order to determine the actual concentration of nitrous oxide present, for example a concentration of 100 ppm.

One way to avoid the cross-interference described above is to design (configure) the optical gas sensor so that its measurement wavelength or measurement wavelength range is in the range of 4.5-4.6 μm. For this purpose, for example, an optical filter can be provided upstream of the radiation detector, which is configured to transmit only wavelengths in the aforementioned range, since nitrous oxide has a measurable absorption in this range, but carbon dioxide has essentially no absorption in this range.

The accuracy of the process according to the invention can be improved if the nitrous oxide concentration is not corrected with a predetermined reference value, but is corrected using an actual carbon dioxide concentration of the breath sample. For this purpose, the gas measuring device can, for example, have a further optical gas sensor which is configured to determine the carbon dioxide concentration. Alternatively, the optical gas sensor can, for example, have a further radiation detector which is configured to measure the carbon dioxide concentration. Alternatively, in the event that only a radiation detector is provided, the optical gas sensor can, for example, have a tunable filter in order to alternately measure nitrous oxide and carbon dioxide. Further embodiments for setting up the gas measuring device to determine the carbon dioxide concentration are possible.

These and other features and advantages can also be seen from the following description of the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing an embodiment of a gas measuring device (breathalyzer) according to the invention;

FIG. 2 is a schematic view showing another embodiment of a gas measuring device (breathalyzer) according to the invention; and

FIG. 3 is flow diagram showing an example of a process according to the invention

### DESCRIPTION OF PREFERRED EMBODIMENTS

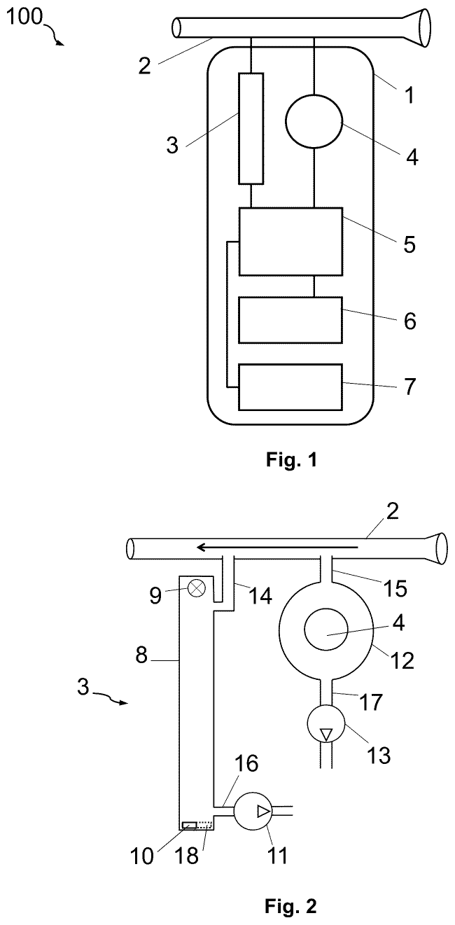

Referring to the drawings, as shown in FIGS. 1 and 2, a mobile or portable gas measuring device (breathalyzer) 100 for determining the concentration of nitrous oxide and ethanol in a breath sample of a test person is provided according to the invention.

The gas measuring device 100 has an optical gas sensor 3 for determining a nitrous oxide concentration in the breath sample and an electrochemical gas sensor 4 for determining an ethanol concentration in the breath sample.

The gas measuring device 100 can also have further elements, as shown in FIG. 1. For example, the gas measuring device 100 can also have a control unit 5 for controlling and evaluating the measuring process, a display unit 6 and an energy storage unit 7 for supplying the electrical and electronic components of the measuring device 100 with energy. The aforementioned components can be connected to the optical gas sensor 3 and/or the electrochemical gas sensor 4 in terms of energy and/or signals, for example in order to obtain measurement signals from the respective gas sensors 3, 4. All or some of the aforementioned components can be surrounded by a common housing 1.

As shown in FIG. 1, the gas measuring device 100 can also have a sample guide element 2, which can be connected to the optical gas sensor 3 and to the electrochemical gas sensor 4 in terms of flow in order to guide the breath sample to the optical gas sensor 3 and to the electrochemical gas sensor 4. As is also shown, the sample guide element 2 can be configured as a mouthpiece 2. The housing 1 of the gas measuring device 100 can have one or more interfaces for receiving the mouthpiece 2, so that it can be connected to the gas measuring device 100 in a replaceable manner.

FIG. 2 shows a detailed view of a gas detection device 100 according to the invention, which may be a detailed view of the gas detection device 100 according to FIG. 1 or may represent an independent embodiment.

FIG. 2 shows that the optical gas sensor 3 can have an optical cuvette 8. The optical cuvette 8 can optionally be heated. The optical gas sensor 3 can also have a radiation source 9 and a radiation detector 10 or several radiation detectors. The sample guide element 2 and the optical cuvette 8 can be fluidically connected by means of a line 14 in order to guide (conduct) the breath sample to the optical gas sensor 3.

The electrochemical gas sensor 4 can be arranged in a sensor holder (mount) 12, which can be fluidically connected to the sample guide element 2 via a line 15 in order to guide the breath sample to the electrochemical gas sensor 4. Line 14 and line 15 can, as shown, be connectable or connected to the sample guide element 2 at two different points or, as not shown, can be connectable or connected to the sample guide element 2 at the same point.

FIG. 2 shows that the gas measuring device 100 can also have a pump unit 11, which is configured to convey at least part of the breath sample to the optical gas sensor 3. In the preferred example shown, the pump unit 11 is arranged downstream of the optical gas sensor 3 and is connected to the sample guide element 2 via line 16, optical cuvette 8 and line 14. In this way, any gas storage behavior of the pump unit 11 for the target gas is negligible. However, any other arrangement of the pump unit 11 relative to the optical gas sensor 3 is also possible as long as the pump unit 11 can deliver the breath sample to the optical gas sensor 3. The pump unit 11 can be configured as a diaphragm pump or as a piezo pump.

FIG. 2 shows that the gas measuring device 100 can also have a further pump unit 13, which is configured to convey at least a further part of the breath sample to the electrochemical gas sensor 4. In the example shown, the further pump unit 13 is arranged downstream of the electrochemical gas sensor 4 and is connected to the sample guide element 2 via line 17, sensor holder 12 and line 15. However, any other arrangement of the pump unit 13 relative to the electrochemical gas sensor 4 is also possible as long as the pump unit 13 can convey the breath sample to the electrochemical gas sensor 4. The further pump unit 13 can be configured as a bellows pump.

The sample guide element 2 can be configured to guide the entire breath sample into the gas measuring device 100 or to guide only part of the breath sample and a further part of the breath sample into the gas measuring device 100 and release the remaining part into the environment, as indicated by an arrow shown in the sample guide element 2 in FIG. 2.

It is not shown that only a pump unit 11 can be provided, which can be connected to both line 16 and line 17 in order to convey at least a portion of the breath sample to the optical gas sensor 3 and at least another portion of the breath sample to the electrochemical gas sensor 4.

The pump unit 11 or the further pump unit 13 can release the breath sample delivered to the respective gas sensor 3 and/or 4 into the environment of the gas measuring device 100 after analysis.

The optical gas sensor 3 can have a measurement wavelength of 4.44 μm to 4.65 μm.

The optical gas sensor 3 can have an optical filter for selecting the measurement wavelength, whereby the optical filter can be exchangeable in order to adapt the optical gas sensor 3 to different target gases. The optical filter or the optical filter that is configured to be replaceable to adapt the optical gas sensor to different target gases or is tunable to selectively change the optical filter to adapt the optical gas sensor to different target gases and is provided upstream of the radiation detector (10, 18) is shown schematically as associated with the radiation detector (10, 18) in FIG. 2.

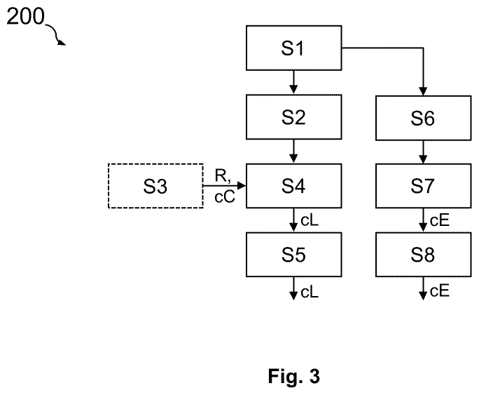

FIG. 3 shows an embodiment of a process 200 according to the invention for determining the concentration of nitrous oxide and ethanol in a breath sample from a test person.

Step S1 is the provision of the gas measuring device 100.

Step S2 is the feeding of at least part of the breath sample to the optical gas sensor 3. The feeding step may comprise a simple guiding of the expelled breath sample to the optical gas sensor 3 or may comprise a conveying of the breath sample to the optical gas sensor 3 by means of the pump arrangement as described.

The optional step S3 is to obtain a reference value R, which indicates a carbon dioxide concentration cC in the breath sample.

Step S4 is the determination of a nitrous oxide concentration cL based on measurement signals from the optical gas sensor 3 and optionally taking into account the reference value R.

Step S5 is the provision of the determined nitrous oxide concentration cL.

Step S6 is the feeding of at least a further portion of the breath sample to the electrochemical gas sensor 4. The feeding step may comprise a simple guiding of the expelled breath sample to the electrochemical gas sensor 4 or may comprise a conveying of the breath sample to the optical gas sensor 3 by means of the pump arrangement as described.

Step S7 is the determination of an ethanol concentration cE based on measurement signals from the electrochemical gas sensor 4.

Step S8 is the provision of the specific ethanol concentration cE.

As indicated, steps S2 and S6 can run simultaneously. However, this is not necessary. It is possible, for example, that step S2 is carried out continuously during the entire measurement process, i.e. that at least part of the breath sample is continuously passed to the optical gas sensor in order to determine the nitrous oxide concentration. Furthermore, for example, it is possible that step S6 is only carried out at a late point in time or during a late period of the entire measurement process, so that a breath sample exiting at the beginning of an exhalation process and thus possibly mouth alcohol is not measured, but a breath sample exiting at the end of an exhalation process and thus lung alcohol corresponding to deep lung air is measured. In this respect, the measurement process can correlate with the duration of the exhalation process. The actual measurement process can be preceded by a zero-point determination of the nitrous oxide concentration and the ethanol concentration.

Preferably, the reference value R is a predetermined reference value R. Additionally, or alternatively, it is preferred that the reference value R is obtained by measuring the carbon dioxide concentration cC in the breath sample by means of the gas measuring device 100.

Some or all of the steps of the above-described process 200 may be performed by means of the control unit 5 of the gas measuring device 100. Steps S5 and/or S8 may comprise displaying the nitrous oxide concentration cL and/or the ethanol concentration cE on the display unit 6 of the gas measuring device 100. Furtherly or alternatively, steps S5 and/or S8 may comprise transmitting the nitrous oxide concentration cL and/or the ethanol concentration cE to an external receiving unit, such as a PC or a separate hand-held device, such as a smartphone or a tablet.

While specific embodiments of the invention have been shown and described in detail to illustrate the application of the principles of the invention, it will be understood that the invention may be embodied otherwise without departing from such principles.

### LIST OF REFERENCE CHARACTERS

1 Housing
2 Mouthpiece, sample feeding element
3 Optical gas sensor
4 Electrochemical gas sensor
5 Control unit
6 Display unit
7 Energy storage
8 Sample chamber, optical cuvette
9 Radiation source
10 Radiation detector
11 Pump unit
12 Sensor holder
13 Further pump unit
14-17 Cables
18 Further radiation detector
100 Gas measuring device
200 Process
cC Carbon dioxide concentration
CE Ethanol concentration
CL Nitrous oxide concentration
R Reference value
S1, S2, . . . Process steps

What is claimed is:

1. A mobile or portable gas measuring device for determining a concentration of nitrous oxide and ethanol in a breath sample of a test person, the gas measuring device comprising:
an optical gas sensor configured to determine a nitrous oxide concentration in the breath sample; and
an electrochemical gas sensor configured to determine an ethanol concentration in the breath sample.

2. A gas measuring device according to claim 1, further comprising a sample guide element configured to be fluidically connected to the optical gas sensor and to the electrochemical gas sensor and to guide the breath sample to the optical gas sensor and to the electrochemical gas sensor.

3. A gas measuring device according to claim 2, further comprising an optical cuvette.

4. A gas measuring device according to claim 3, wherein the optical cuvette is configured to be heated.

5. A gas measuring device according to claim 1, wherein the optical gas sensor comprises:
a radiation source, and
a radiation detector.

6. A gas measuring device according to claim 5, wherein the optical gas sensor further comprises a further radiation detector.

7. A gas measuring device according to claim 2, wherein the sample guide element is configured as a mouthpiece.

8. A gas measuring device according to claim 1, further comprising a pump unit which is configured and arranged to convey at least a portion of the breath sample to the optical gas sensor and/or to the electrochemical gas sensor.

9. A gas measuring device according to claim 8, further comprising a further pump unit which is configured and arranged to convey at least a further portion of the breath sample to the electrochemical gas sensor and/or to the optical gas sensor.

10. A gas measuring device according to claim 8, wherein the pump unit is configured as a bellows pump.

11. A gas measuring device according to claim 9, wherein the pump unit and/or the further pump unit is configured as a bellows pump.

12. A gas measuring device according to claim 5, wherein the optical gas sensor has a measurement wavelength in a range from 4.44 μm to 4.65 μm.

13. A gas measuring device according to claim 5,
wherein the optical gas sensor comprises an optical filter for selecting a measurement wavelength, and
wherein the optical filter is configured to be replaceable to adapt the optical gas sensor to different target gases.

14. A process for determining a concentration of nitrous oxide and ethanol in a breath sample of a subject, the process comprising the steps of:
providing a mobile or portable gas measuring device, the gas measuring device comprising: an optical gas sensor configured to determine a nitrous oxide concentration in the breath sample; and an electrochemical gas sensor configured to determine an ethanol concentration in the breath sample;
feeding at least part of the breath sample to the optical gas sensor;
determining a nitrous oxide concentration based on measurement signals from the optical gas sensor;
providing the determined nitrous oxide concentration;
feeding at least a further portion of the breath sample to the electrochemical gas sensor;
determining an ethanol concentration based on measurement signals from the electrochemical gas sensor; and
providing the determined ethanol concentration.

15. A process according to claim 14, further comprising the step of obtaining a reference value which indicates a carbon dioxide concentration in the breath sample, wherein the determination of the nitrous oxide concentration is also based on the obtained reference value.

16. A process according to claim 14, wherein the reference value is a predetermined reference value, or the reference value is obtained by measuring the carbon dioxide concentration in the breath sample with the gas measuring device.

17. A breathalyzer for determining both a concentration of nitrous oxide and ethanol in a breath sample of a test person, the breathalyzer comprising:
an optical gas sensor configured to determine a nitrous oxide concentration in the breath sample, the optical gas sensor comprising a radiation source, and a radiation detector; and
an electrochemical gas sensor configured to determine an ethanol concentration in the breath sample, wherein the breathalyzer is configured to be mobile/portable.

18. A breathalyzer according to claim 17, further comprising:
an optical cuvette associated with the optical gas sensor; and
a sample guide element configured to be fluidically connected to the optical gas sensor and to the electrochemical gas sensor and to guide the breath sample to the optical cuvette of the optical gas sensor and to the electrochemical gas sensor.

19. A breathalyzer according to claim 18, further comprising a pump arrangement comprising a pump unit configured and arranged to convey at least a portion of the breath sample to the electrochemical gas sensor and/or to the optical gas sensor or comprising a pump unit configured and arranged to convey at least a portion of the breath sample to the electrochemical gas sensor and another pump unit configured convey at least a portion of the breath sample to the optical gas sensor.

20. A breathalyzer according to claim 18,
wherein the optical gas sensor comprises an optical filter for selecting a measurement wavelength, and
wherein the optical filter is configured to be replaceable to adapt the optical gas sensor to different target gases or tunable to selectively change the optical filter to adapt the optical gas sensor to different target gases.
